(19) United States
(12) Patent Application Publication
(10) Pub. No.: US 2017/0189634 A1
(43) Pub. Date: Jul. 6, 2017

(54) System, Method, and Device For Airway Assessment and Endotracheal Intubation

(71) Applicant: Barrett J. Larson, Palo Alto, CA (US)

(72) Inventor: Barrett J. Larson, Palo Alto, CA (US)

(21) Appl. No.: 14/714,189

(22) Filed: May 15, 2015

Related U.S. Application Data
(60) Provisional application No. 61/993,275, filed on May 15, 2014.
(60) Provisional application No. 62/104,682, filed on Jan. 16, 2015.
(60) Provisional application No. 62/117,461, filed on Feb. 18, 2015.

(51) Int. Cl. A61M 16/04; A61B 5/00; A61B 5/05; A61B 8/08; A61B 8/00

(52) U.S. Cl. CPC A61M 16/0488; A61B 8/085; A61B 8/42; A61B 8/461; A61B 5/05; A61B 5/4887; A61B 5/742

(57) ABSTRACT

An airway assessment and intubation guidance system, device and method that is portable, simple to use, and effective in reliably locating the trachea within the neck of a wide variety of patients and independent of fluids that may be found within the trachea. The system permits an objective assessment of the likelihood of a difficult intubation, and further provides, depending upon the embodiment, both visual and audible feedback regarding the location of a compatible stylet relative to the trachea.

### RELATED APPLICATIONS

This application is a continuation-in-part application of U.S. patent application Ser. No. 14/714,189 filed May 15, 2015, and through it further claims the benefit of, and priority from, the following U.S. provisional patent applications: Ser. 61/993,275, filed May 15, 2014; Ser. 62/104,682 filed Jan. 16, 2015, and Ser. 62/117,461, filed Feb. 18, 2015, all of which are incorporated herein by reference for all purposes.

### FIELD OF THE INVENTION

This invention pertains generally to intubation and airway assessment devices and methods. More specifically, this invention pertains to an intubation means that does not require direct visualization of the vocal cords, trachea, or other airway landmarks. In addition, the invention comprises devices and methods for making a non-invasive, objective assessment of a patient's airway and the expected difficulty of intubation.

### BACKGROUND OF THE INVENTION

Airway assessment in anticipation of a tracheal intubation in a patient is an important skill for an anesthesiologist. There are a variety of prior art risk assessment tools designed to identify difficult airways, but they all have limited specificity and sensitivity. One of the most commonly used tools is the Mallampati test, but this has been shown to have a poor sensitivity and specificity. There exists a need for an objective, convenient and reliable means for identifying patients that may have a difficult airway so that appropriate safety measures can be taken prior to inducing general anesthesia and attempting to intubate the patient.

Once an airway assessment is made, intubation typically follows. Conventional methods for endotracheal intubation involve direct visualization of the vocal cords. A laryngoscope is a commonly used instrument that allows an operator to insert an endotracheal tube through the vocal cords under direct vision. However, use of a laryngoscope often requires neck manipulation, which may be contraindicated in the setting of a known or suspected neck injury. Furthermore, variations in the airway anatomy may make direct visualization of the vocal cords difficult or impossible.

Other common means for endotracheal intubation involve indirect vocal cord visualization, such as by video laryngoscopy or fiberoptic imaging. These devices can be effective alternatives to direct laryngoscopy, but each technique has its own set of limitations. For example, limitations in mouth opening can make video laryngoscopy difficult or impossible. Additionally, if the airway has been compromised by blood, secretions, or gastric contents it may be difficult or impossible to visualize the vocal cords using any optical means. Finally, fiberoptic and video laryngoscopes are generally larger pieces of equipment that are not easily transportable, such as in a pocket.

Another method for endotracheal intubation involves using a lighted stylet. With this method, a stylet having a light source at the distal tip is used to help guide endotracheal tube placement. The light source transilluminates through the tissues of the neck, such that the stylet can be guided into the trachea. Successful insertion of the stylet into the trachea is, in ideal cases, represented by a characteristic light pattern that is visible through the tissues of the anterior neck. This intubation method does not require neck manipulation, which is desirable in the setting of a suspected or known neck injury. Furthermore, the technique is relatively insensitive to blood or other fluids that may impair visualization of the vocal cords. Finally, given the low profile nature of the device, this technique is less likely to cause tissue trauma or dental injury. There is also evidence to suggest that this technique produces less sympathetic stimulation than direct laryngoscopy and provides improved hemodynamic stability during intubation.

However, as with other intubation methods, the transillumination technique does have certain limitations. First, the technique generally requires a dimly lit or dark environment, which may not always be feasible, such as in the emergency room or the pre-hospital setting. Second, the transillumination technique can be more difficult in obese patients, where there is a large amount of soft tissue in the anterior neck. In extremely thin patients, the opposite problem exits, and the characteristic pre-tracheal glow may be seen even with an esophageal intubation. The transillumination technique may also be more difficult in patients with darker skin tones. Finally, thermal injury may occur if tissues are exposed to the light source for a prolonged period of time.

There are many methods and devices used for confirming that an endotracheal tube has been properly placed. One method is to look for bilateral chest rise upon ventilating the patient and listening for breath sounds. Another highly reliable method for confirming proper endotracheal tube placement is to look for exhaled carbon dioxide upon ventilating the patient. These techniques can also be used to monitor for inadvertent extubation. For example, an abrupt loss of exhaled carbon dioxide can indicate that the endotracheal tube is no longer in the trachea.

Another method that has been described for confirming endotracheal tube placement involves magnetic field sensing. With this method, a small magnet is embedded into the endotracheal tube, such that the magnet can be detected via a magnetic sensing element that is placed external to the body. The intensity of the detected magnetic field is intended to correlate with the position of the magnet so that the relative position of the endotracheal tube can be determined. In order to confirm initial endotracheal tube placement, the sensor attempts to determine if the detected endotracheal tube depth is compatible with the expected depth of the trachea. Once intubation has been confirmed, if the relative position of the endotracheal tube moves significantly, care providers can be notified that the endotracheal tube may no longer be in the correct position. A major limitation of this technique is the inability for these devices to accurately determine the true location and boundary limits of the trachea.

Previously described methods attempt to use magnetic field sensing to probabilistically determine if the three-dimensional location of an endotracheal tube is likely compatible with endotracheal placement. These determinations are based on the expected tracheal depth for an average patient. If the endotracheal tube is determined to be at a depth that is greater than the expected depth of the trachea, then esophageal intubation is suspected. However, it is well known that the depth of the trachea and the cross-sectional diameter of the trachea can vary widely among patients. For example, in obese patients, the trachea can lie over three centimeters beneath the skin surface. On the contrary, in thin patients, the trachea can be located just a fraction of a centimeter beneath the skin surface. The variability that exists in tracheal depth is greater than the average cross-sectional diameter of the trachea. Therefore, without knowing the true depth and location of the trachea for a given patient, magnetic field sensing alone is unlikely to reliably confirm endotracheal tube placement. Given that confirming proper endotracheal tube placement is a life-critical assessment, it is important for such devices to reliably indicate to a caregiver whether the magnet is contained within the boundaries of the trachea, regardless of where the trachea may be located.

If a device could provide real-time information regarding the location of the endotracheal tube in relation to the trachea and other anatomic landmarks, that information could facilitate the intubation process. A guidance system that allows care providers to intubate the trachea using at least real-time visual feedback would offer significant benefits over the prior art.

### SUMMARY OF THE INVENTION

The present invention comprises an airway assessment and intubation guidance system, device and method that is portable, simple to use, and effective in reliably locating the trachea within the neck of a wide variety of patients and independent of fluids that may be found within the trachea. The system permits an objective assessment of the likelihood of a difficult intubation, and further provides, depending upon the embodiment, both visual and audible feedback regarding the location of a compatible stylet relative to the trachea. Neck circumference, which is useful in locating the trachea, can be determined in the present invention through the use of a plurality of accelerometers properly positioned on a substrate draped around part of all of a patient's neck. The accelerometers provide data to a controller or microprocessor which can then algorithmically determine neck circumference. The device can further interface to a software application for display on a processor-based device, such as a smartphone, computer, or the like.

An application of the present invention is to provide an improved means for endotracheal intubation that incorporates magnetic field sensing and a means for localizing the trachea and other anatomic landmarks in real time. The device of the present invention can comprise a plurality of magnetometers or other magnetic sensors for identifying the location of a magnetic stylet relative to the trachea. By determining the location of both a magnetic intubation stylet and the trachea, the invention described herein provides a more reliable intubation guidance system than prior art devices. The device of the present invention can also comprise, in some embodiments, one or more ultrasound transducers and receivers to further identify anatomical landmarks internal to the neck, further improving intubation guidance. Alternatively, body-penetrating radar can be used instead of ultrasound or similar devices.

These and other benefits of the present invention can be further appreciated from the following Detailed Description of the Invention, taken in conjunction with the appended Figures.

### FIGURES

FIG. 1 illustrates in plan view a guidance device in accordance with an implementation of the invention.

FIG. 2 illustrates in side elevation view a guidance device in accordance with an implementation of the invention substantially as shown in FIG. 1.

FIG. 3 illustrates the guidance device of FIG. 1 in position around a simplified view of the neck of a patient.

FIG. 4 illustrates a preferred orientation of the guidance device of FIG. 1 relative to the neck.

FIG. 5A illustrates the interaction of an intubation stylet and the guidance device in accordance with an embodiment of the invention.

FIG. 5B illustrates the stylet-guidance device interaction as the stylet moves closer to the desired position for a successful intubation.

FIG. 5C illustrates the stylet-guidance device interaction as the stylet moves to a central position relative to the guidance device.

FIG. 5D illustrates the stylet-guidance device interaction as the stylet is properly positioned for tracheal intubation.

FIG. 6 illustrates a stylet having a magnetic aspect in accordance with an embodiment of the invention.

FIG. 7A illustrates an alternative embodiment of the guidance device.

FIG. 7B illustrates a still further alternative embodiment of the guidance device.

FIG. 8 illustrates in schematic diagram form the key elements of an embodiment of the guidance device such as shown in FIG. 7A.

FIG. 9 illustrates in flow diagram form the operation of an embodiment of the guidance device in response to proximate movement of a stylet in accordance with the invention.

FIGS. 10A-10C illustrate in flow diagram form various embodiments of techniques for assessment of a patient with respect to potential intubation issues.

FIG. 11 illustrates the calculation of neck diameter.

### DETAILED DESCRIPTION OF THE INVENTION

Described herein is a means for endotracheal intubation that overcomes many of the limitations of the prior art. The guidance apparatus (“the apparatus”) is placed on, near, or around the patient's neck. In some implementations, the apparatus can be placed in a known orientation relative to the trachea. For example, the center of the apparatus may be placed in the midline of the patient's neck, directly over the thyroid cartilage. The orientation of the apparatus with respect to the patient can be defined or determined. In some implementations, the apparatus has markings, or a characteristic shape, or another means for indicating how to properly orient the device with respect to the patient. In some implementations, the orientation of the apparatus with respect to the patient is automatically determined. In some implementations, proper use of the device does not depend on its orientation with respect to the patient, but rather the device can automatically determine the depth and location of the trachea and other anatomic landmarks relative to the device regardless of device orientation.

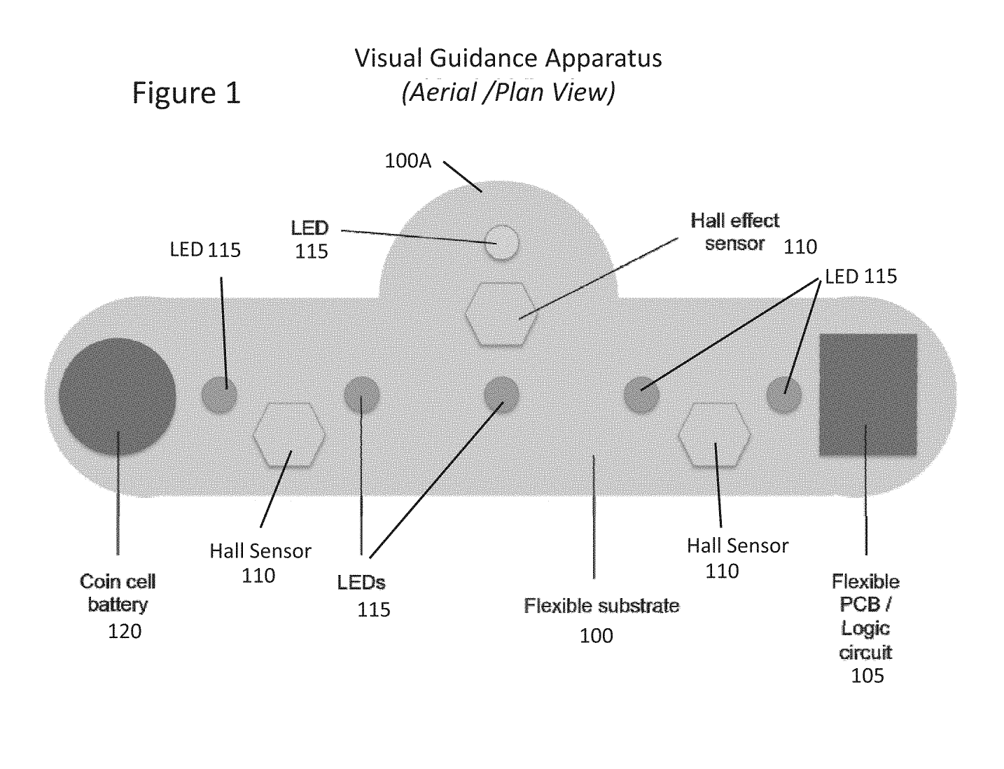

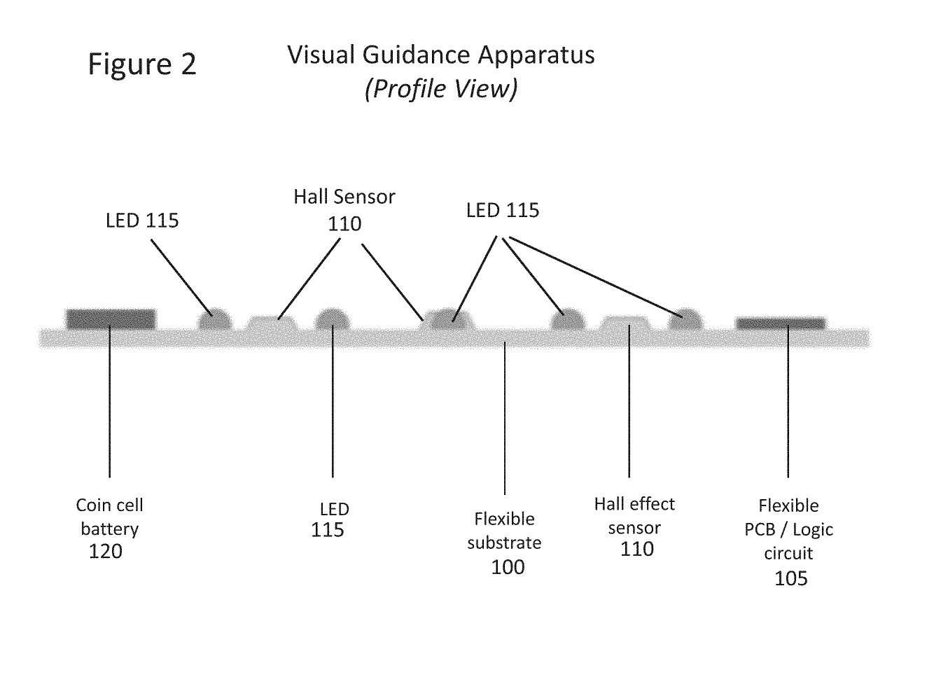

With reference to FIGS. 1-3, the guidance apparatus comprises a substrate 100 on which is mounted a controller or processor 105 together with other appropriate logic as will be understood by those skilled in the art. A tab 100a of the substrate can be provided in some embodiments. The tab provides an asymmetry to the substrate to help facilitate proper orientation of the substrate with respect to the patient, but is not required in all embodiments, and can be sized to permit comfortable placement of the substrate on the neck. The guidance apparatus can further comprise one or more sensors 110 that are responsive to a magnetic field and provide their outputs to the controller 105. Possible sensors 110 include Hall effect sensors, 3-axis Hall sensors, magnetometers, electronic compass, reed switches, fluxgate magnetometers, magnetoresistance-based sensors, or other magnetic field or proximity sensors. Throughout this disclosure, the term “magnetometer” is used for clarity of disclosure, but is intended to encompass any suitable magnetic or electric field sensor. In some embodiments, the sensors 110 can include accelerometers for assisting in the determination of neck circumference, as discussed in greater detail hereinafter. The accelerometers and magnetic sensors can, but need not, be implemented in the same electronic chip.

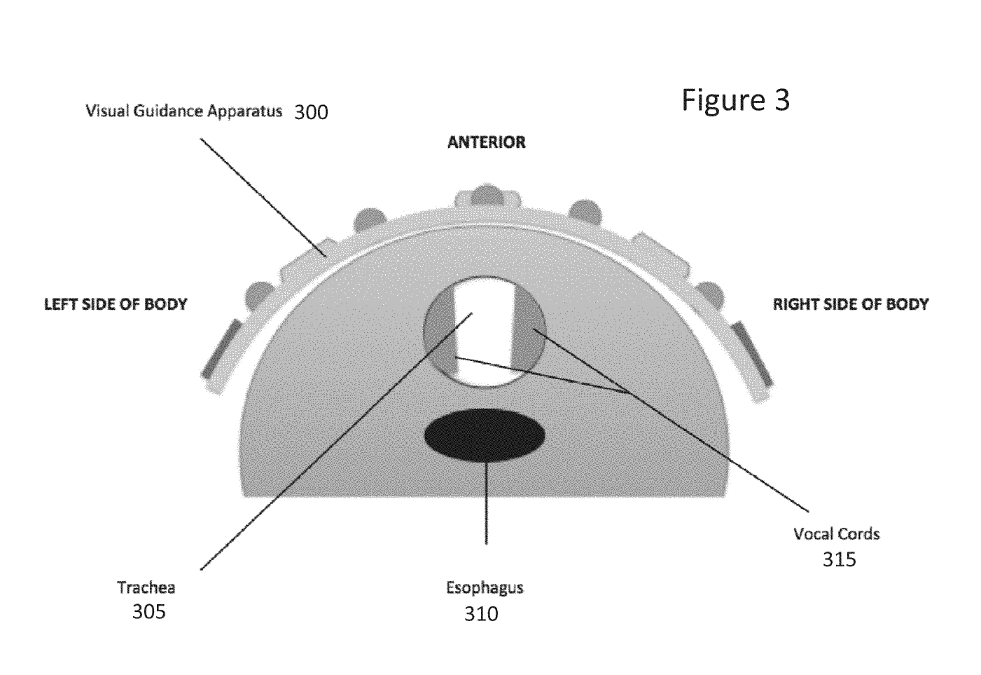

In an embodiment, at least a portion of the magnetic guidance apparatus extends directly over the trachea, such as shown particularly in FIG. 3. It will be appreciated by those skilled in the art that the trachea 305 is positioned anterior to the esophagus 310, with the vocal cords 315 positioned proximate to the entry to the trachea. In alternative embodiments, the apparatus can be placed over the neck in any orientation and the orientation of the apparatus relative to the patient is automatically determined. In such a fashion, the sensitivity and specificity of detecting endotracheal placement of the magnetic intubation stylet can be increased.

The curvature of a patient's neck and the degree of soft tissue compliance can vary widely among patients. In some implementations, the apparatus will be flexible enough to bend and conform to the curved surface of the patient's neck. The apparatus can be built on a flexible substrate, such as a polyimide, conductive fabric, or any other suitable material. In some implementations, the apparatus may be wrapped completely around the neck or around a portion of the neck. In still other implementations, the apparatus may not be in direct contact with the patient, but instead is remotely associated with the patient in a known orientation/location relative to the patient. In other implementations, the substrate can comprise a material having memory, such that, once positioned on a patient's neck, the shape of the neck is preserved at least somewhat by the memory characteristic of the material, thus helping to ensure proper placement and retention on the anterior surface of the next. As described hereinafter, in some embodiments the apparatus may contain a sensing means to determine the extent to which the apparatus is flexed, bent, wrapped around, or is otherwise associated with the patient's neck or other tissues. Furthermore, in some embodiments the apparatus can contain a means for determining the degree of contact with a subject's skin, such as by capacitive or thermal sensing or other means. In some implementations, the device can provide user feedback to indicate whether the apparatus is making sufficient contact with the patient, or the device can automatically calibrate itself based on the degree to which the apparatus is contacting the patient.

In some implementations, the output of the sensor(s) is related to the magnitude and direction of an externally applied magnetic field. In at least some embodiments, the apparatus contains an array of magnetometers. In some implementations, the magnetometers are arranged in different relative orientations, which may be predefined or determined. In some implementations, the apparatus has a curved structure or is flexible. When arranged along an arc or a curve, a three-dimensional magnetic sensor array is created. By orientating the magnetometers in a partially or completely circumferential pattern around the target, shown in FIG. 3, the relative location, orientation, and trajectory of an externally applied magnet can be accurately determined. Having a magnet with a known orientation can help users align the long-axis of the magnetic stylet with the long-axis of the trachea in order to facilitate endotracheal intubation. In some embodiments, particularly if single-axis magnetometers are used, some magnetometers may be placed orthogonally to others.

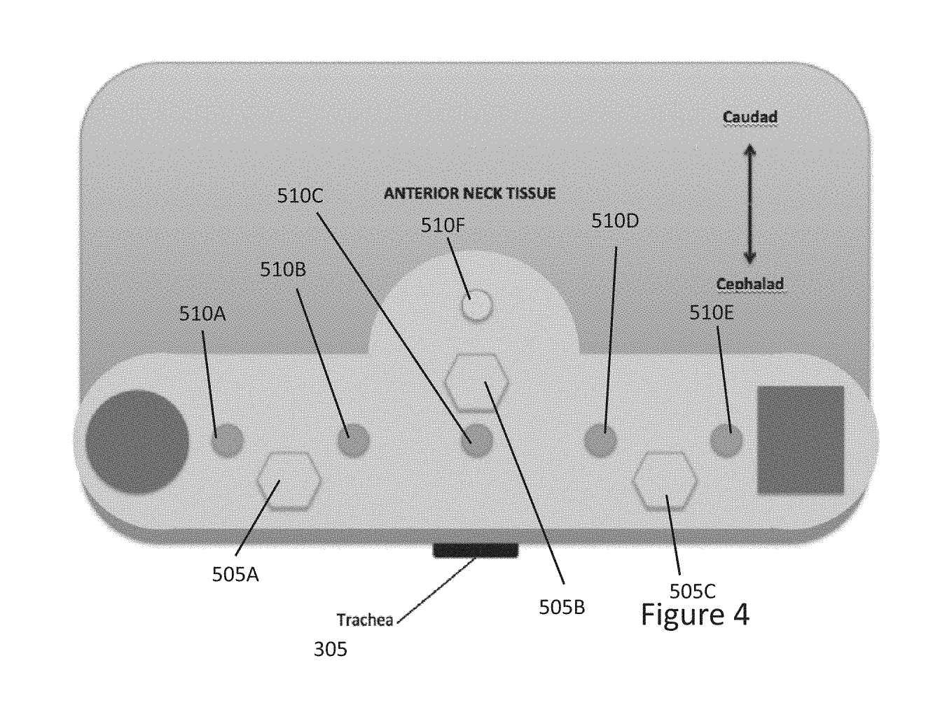

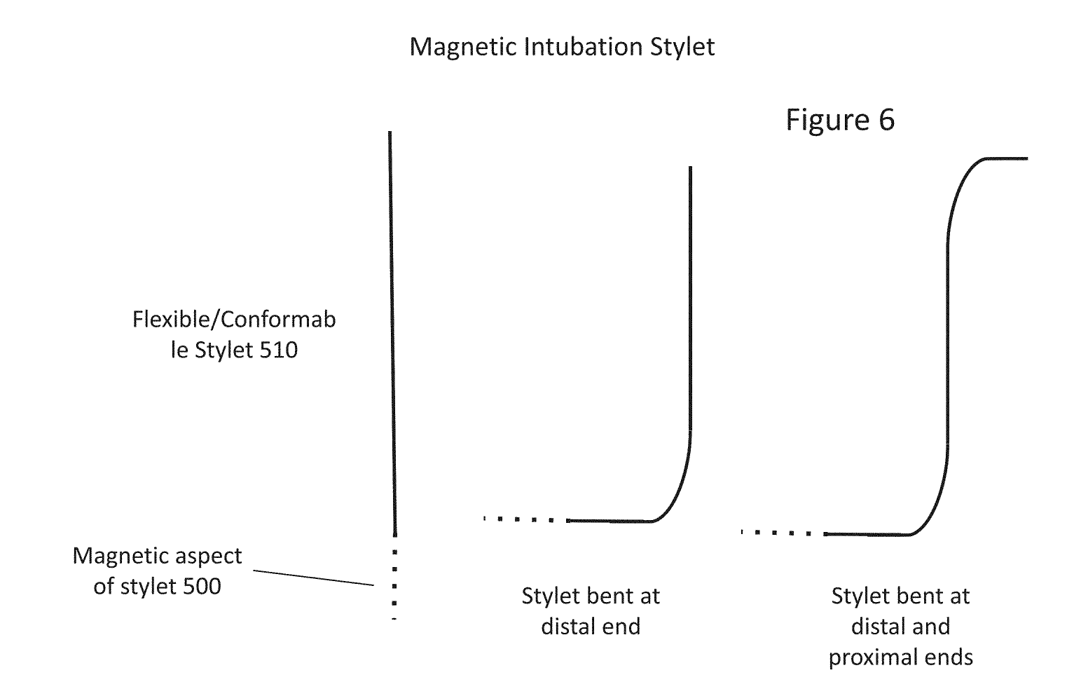

With a magnetic intubation stylet that has a known magnetic field, as shown in FIG. 6, the distance and orientation of the magnetic stylet to individual sensors in the array of magnetometer(s) can be determined. By using the plurality of sensors shown in FIGS. 1A-4, for example, the relative position and orientation of the magnetic intubation stylet can be determined.

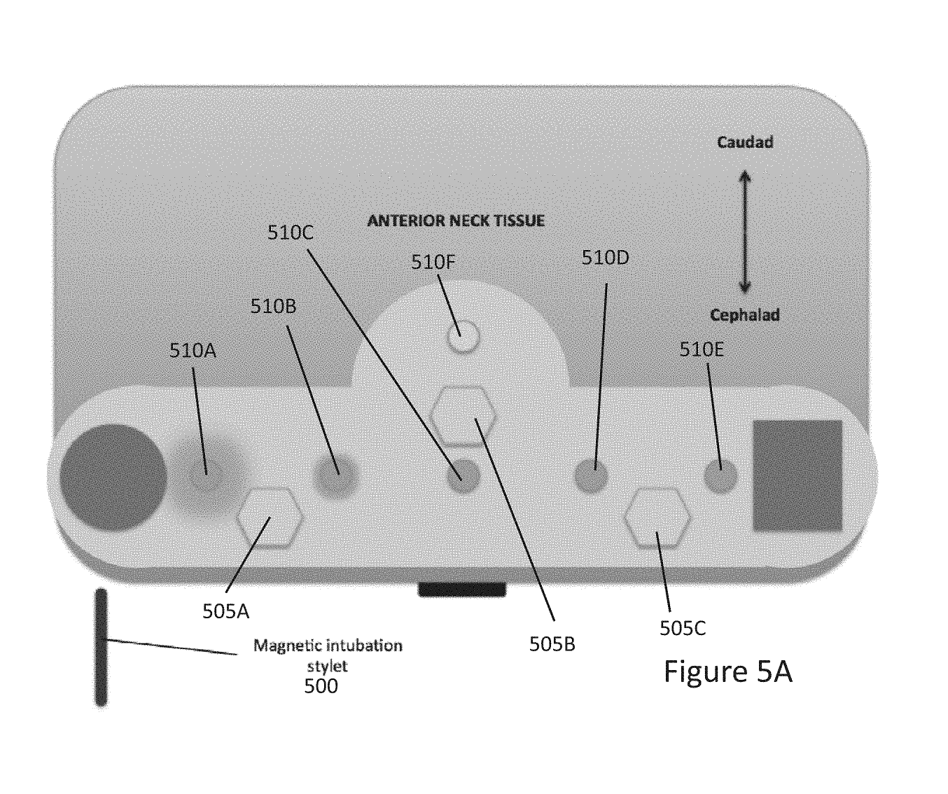

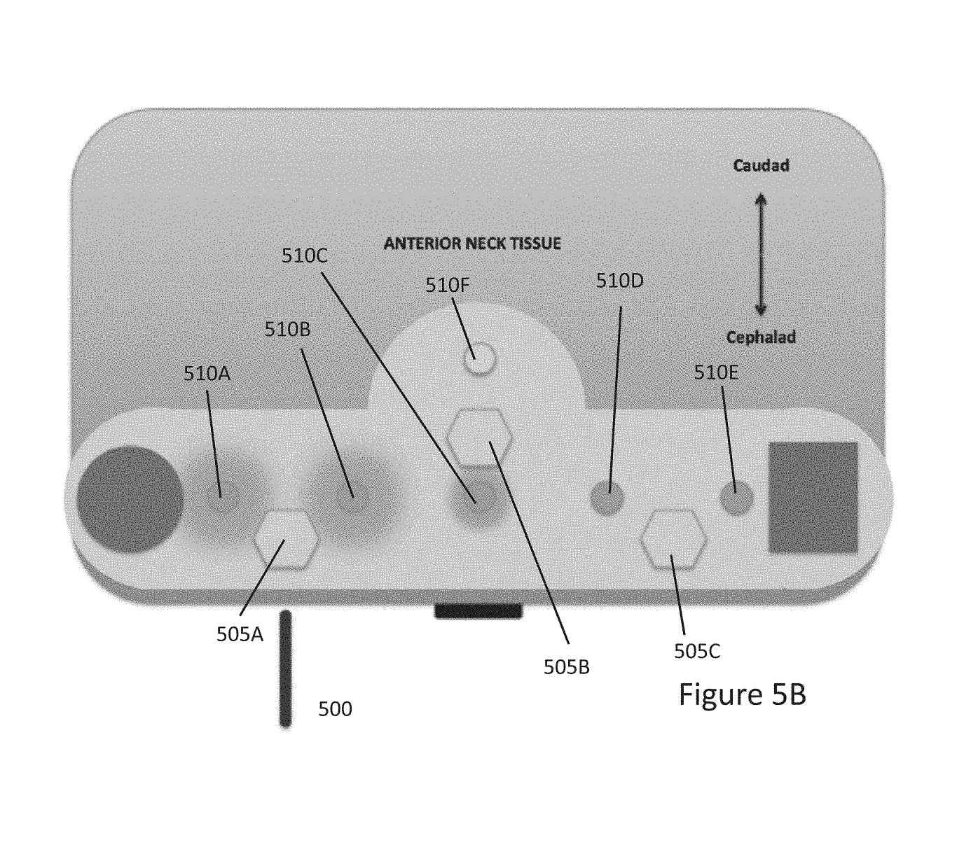

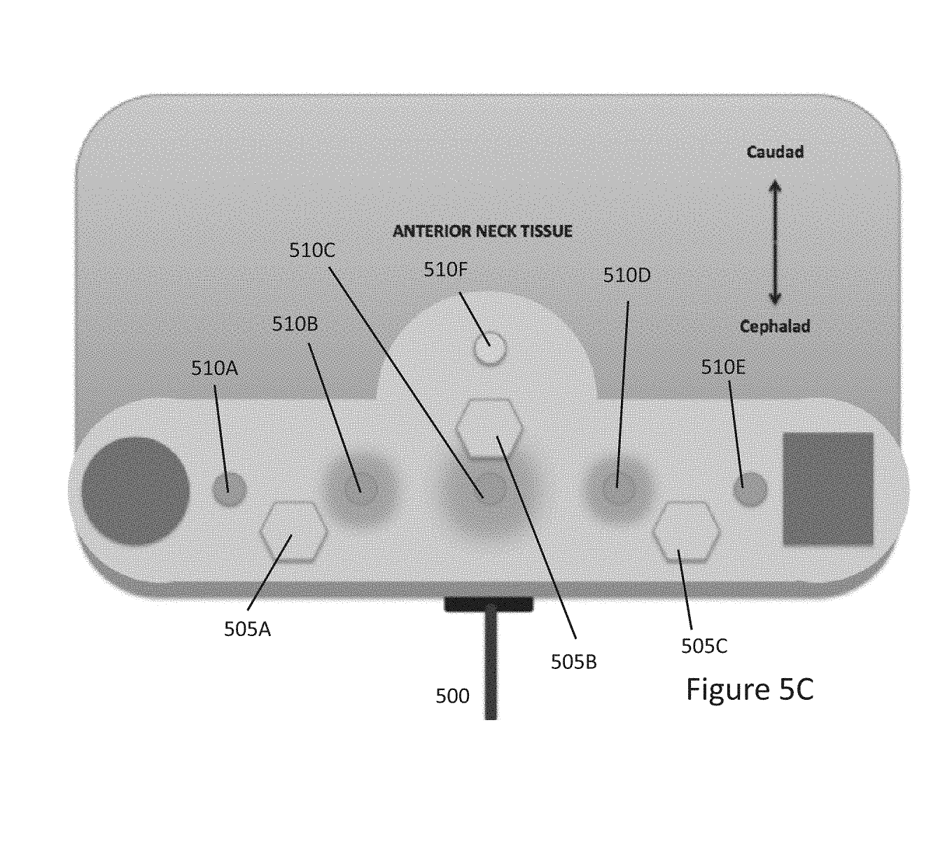

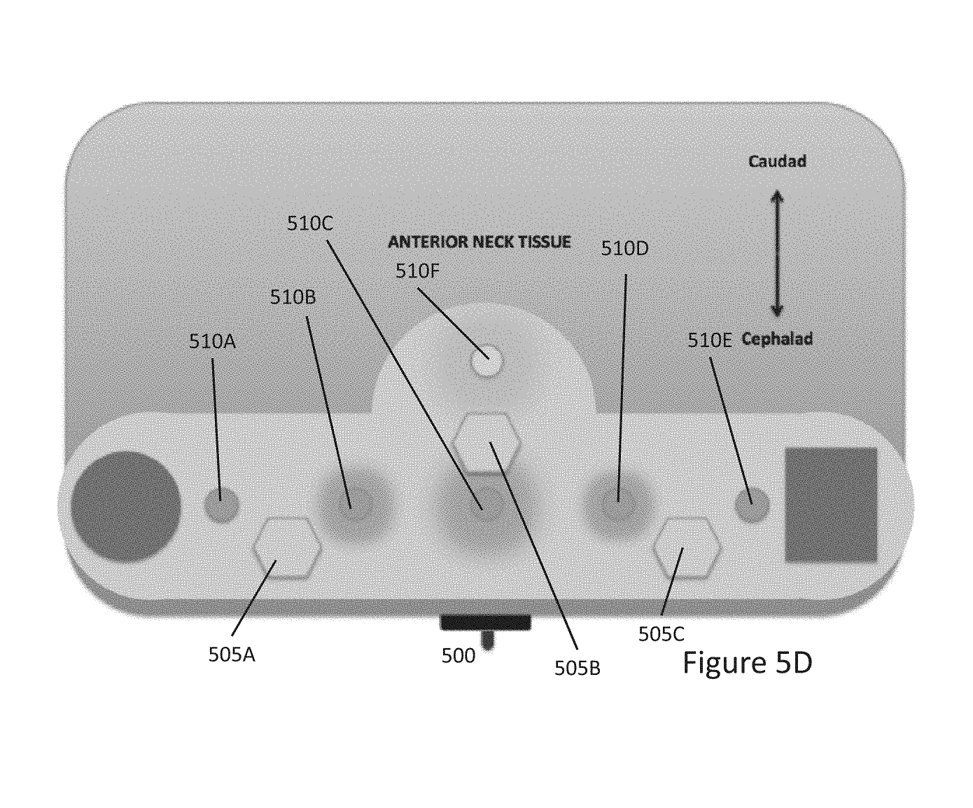

As shown in FIGS. 5A-5D and 6, a magnetic field fingerprint can be used to determine the location, orientation, and trajectory of the magnetic intubation stylet 600 (shown in FIG. 6 and discussed hereinafter) with respect to the LEDs 510A-F on the guidance apparatus. Furthermore, when the stylet is suitably proximate, the magnetic field fingerprint can be defined for all relevant positions and orientations of the individual magnetometer(s). In such a manner, the expected magnetic field fingerprint (as measured by one or more magnetometers) can be defined for all relevant locations and orientations of the magnetic intubation stylet and for every relevant position and orientation of each of the magnetometers. In addition to determining the relative location of the magnetic intubation stylet, the magnetic field fingerprint can permit the controller and sensors to determine the orientation and trajectory of the intubation stylet. As discussed hereinafter, information regarding the position, orientation and trajectory of the intubation stylet relative to the trachea, when provided in real-time, can be used to guide endotracheal intubation.

### Facilitating Endotracheal Intubation

As discussed previously, simply knowing the relative location of the magnetic intubation stylet is not sufficient to reliably guide endotracheal intubation or confirm placement. The guidance apparatus described herein is able to determine the approximate or exact location of the patient's trachea and other anatomic landmarks, which can vary widely between patients. In such a fashion, an improved means for guiding endotracheal intubation is provided.

Depending upon the embodiment, the guidance apparatus can be substantially flexible and able to assume a plurality of shapes. This flexibility allows the apparatus to conform to a variety of different neck shapes and sizes. Given the flexible nature of the apparatus, the curvature of the apparatus can approximate the curvature of the patient's neck. For example, when used on an obese patient with a thick neck and a large neck circumference, the curvature of the apparatus may be relatively shallow. However, on a thin patient with a small neck and a small neck circumference, the curvature of the apparatus may be relatively sharp. The guidance apparatus of the present invention can include a plurality of accelerometers, stretch sensors, strain gauges and/or other sensors to allow the device to measure the curvature of the neck and analyze the patient's topographical surface anatomy.

The measured curvature of the neck provides an indication of the patient's neck circumference. It is known that the circumference of the neck correlates to the depth of the trachea beneath the skin surface. There is a mathematical relationship that defines how tracheal depth correlates to neck circumference. The tracheal depth is related to the neck circumference by the following relationship:

Tracheal Depth=[(Neck Circumference−30 cm)*0.0843]+0.9 cm

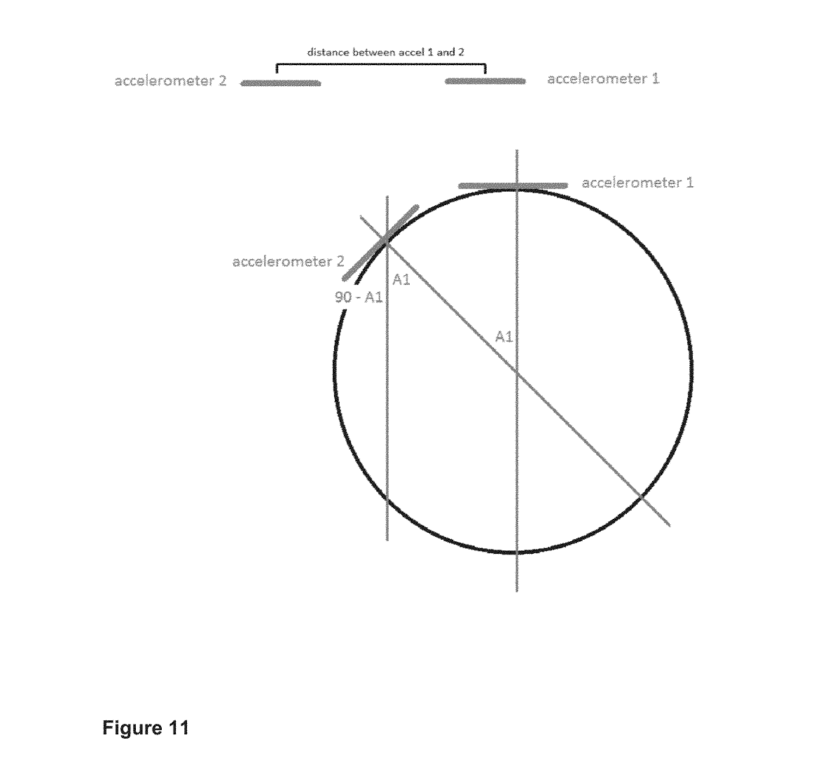

The guidance apparatus of the present invention can utilize two or more accelerometers to determine the patient's neck circumference, and thus tracheal depth. The neck circumference can be calculated from any two accelerometers on the guidance apparatus (A1 and A2), where you have a fixed linear distance between accelerometers A1 and A2, which is equal to distance D1. The neck circumference is defined by the following relationship [see FIG. 11]:

D1/(Neck Circumference)=A1/(360 degrees)

Given that the guidance apparatus of the present invention may have multiple pairs of accelerometers, the calculation of neck circumference can be improved by averaging neck circumference estimates provided by multiple pairs of accelerometers. The tracheal depth can therefore be calculated as follows:

Tracheal Depth=[(D1*(360 degrees)/A1−30 cm)*0.0843]+0.9 cm

By determining the tracheal depth for a patient in real-time, the guidance apparatus can automatically calibrate itself based on the depth of the trachea. In such a fashion, it is possible to indicate the position of a magnetic intubation stylet (or other intubation device) relative to the trachea through proper illumination of LED's located on the device, or, alternatively or additionally, by a visual display shown on a smartphone, monitor, or similar device. Furthermore, the measured surface topography of the neck can provide an indication of the location of relevant anatomic structures. For example, the thyroid cartilage is a prominent landmark located in the midline of the anterior neck. The thyroid cartilage has a characteristic topography that can be detected by the guidance apparatus, and thus the midline of the neck can be identified automatically. Determining the midline of the neck is helpful because the trachea is a midline structure that lies beneath the thyroid cartilage.

The size, shape, curvature, circumference, and surface topography of the neck can provide an indication of the depth and relative location of the trachea beneath the skin surface. It should be noted that the normal transverse diameter of the trachea in adults generally ranges between 15 and 25 mm with a cross-sectional area of 250 to 350 mm². The tracheal diameter correlates with gender, but is not generally associated with body weight, height, or neck circumference. The tracheal depth can be less than 1 cm from the skin surface in thin patients or over 4 cm deep in larger patients. By knowing the expected depth of the trachea (based on neck circumference) and the expected cross-sectional area of the trachea, the boundaries of the patient's trachea can be defined in real-time by the guidance apparatus. Based on the anticipated depth, location, and boundaries of the trachea, the sensitivity of the magnetic sensor array can be optimized either by manual or automatic means. For example, if it is anticipated that the trachea is two centimeters beneath the skin surface, the sensitivity of the magnetic sensor array may be optimized accordingly. Conversely, if it is anticipated that the depth of the trachea is less than 1 cm beneath the skin surface, the sensitivity of the magnetic sensor array may be modulated accordingly. In addition to optimizing the sensitivity of the magnetic sensing array, determining the depth of the trachea can be used to assess whether the magnetic intubation stylet is contained within the trachea, or beneath the trachea (such as in the esophagus). For example, if the trachea is determined to be less than 1 cm from the skin surface, but the magnet is detected 4 cm below the skin surface, the magnet is not located within the trachea. In such a fashion, the visual output of the apparatus can reflect the location of the magnetic intubation stylet relative to the trachea.

As discussed previously, in order for the visual output means to provide an indication of the location of the magnetic stylet relative to the trachea, the guidance apparatus needs to be provided with or to be able to determine or to be able to estimate reasonably the location of the trachea. Successful insertion of the endotracheal tube can only be confirmed when the magnetic intubation stylet is determined to be contained within the boundaries of the trachea. Prior to entering the trachea, the guidance system of the present invention, together with the systems' visual output means, can indicate the depth, laterality, and position of the magnetic stylet with respect to the trachea. If it is determined that the magnetic intubation stylet has entered the esophagus, which is a structure that passes dorsal to the trachea (i.e. deeper than the trachea but in the same plane), this information can be provided via the visual output means. In some implementations, the location of the trachea and magnetic intubation stylet can be represented probabilistically based on the confidence of the measurements obtained by the visual targeting apparatus. Furthermore, the probability of successful insertion of the magnetic intubation stylet into the trachea can be conveyed to a user.

Although neck curvature, circumference, and topographical analysis may provide an adequate means of determining the depth, location, and boundary limits of the trachea, there are other sensing modalities that can be combined or used independently to further resolve the location of the trachea and other anatomic landmarks. In some implementations, the guidance apparatus may incorporate an array of bioimpedance sensors. The impedance sensors can measure the impedance along one or more vectors across the neck. The impedance will change based on the resistivity of intervening tissues.

Electrical Impedance Tomography (EIT) is an impedance-based imaging technique that can be used to analyze a patient's airway anatomy and localize the trachea. Focused Impedance Method (FIM) is a similar technique that uses four or more electrodes to localize a zone of interest within a volume conductor. In an embodiment of the invention, bio-impedance measurements can be obtained from a plurality of electrodes maintained in the device that contact the patient's skin at a plurality of locations when the device is draped across a patient's neck. An embodiment of the invention can include a microprocessor configured to generate a 2-dimensional or 3-dimensional tissue topographic image. The technique leverages differences in the relative impedance/conductivity between different tissue types. For example, the trachea is an air-filled structure (high resistivity) that is contained within a volume conductor that otherwise has a relatively low resistance. Given that the trachea is an air-filled structure, it is a very poor conductor relative to the surrounding tissue and thus there exists a characteristic impedance change that can be differentiated from the surrounding tissue. Electrical Impedance Tomography works by administering a current at plurality of skin surface electrodes to obtain a tissue conductivity map. The electrical potential difference between any two electrodes is related to the current applied, with the impedance being used as the proportionality factor. Algorithms used in FIM or EIT can be used to detect the location of a non-conducting impurity (i.e. trachea/air) within the volume container. A topographical map can be generated with respect to the shape, size, and relative location of the non-conducting impurity (i.e. the trachea).

In addition to determining the location of the trachea, bio-impedance sensing can be used to define the anterior, posterior, and lateral margins of the trachea. Once the boundaries of the trachea have been determined, if a magnetic intubation stylet is determined to be located within these boundaries, then proper endotracheal tube placement can be been confirmed.

Bio-impedance sensing can be influenced by several parameters, including background impedance of patient's neck tissue, depth of the trachea, size, shape, and location of the trachea, expected conductivity of the trachea and surrounding structures, and number and location of bio-impedance electrodes that are used to take measurements. Given that each patient's baseline tissue impedance (i.e. background impedance) will be different, in some implementations absolute values of tissue impedance are not obtained. However, relative changes in impedance across the neck tissue can be detected. Regardless of the background impedance measurements, a characteristic change in impedance will correlate with the location of the trachea. Algorithms that are well known in the art can be used to localize the trachea and other anatomic landmarks based on bio-impedance measurements obtained from a plurality of electrodes.

Another sensing modality that can be used to detect the depth, location, and boundaries of the trachea is acoustic or ultrasonic sensing. An ultrasonic map of the neck can be produced based on the reflection of waves off of the trachea. The strength of the ultrasonic signal and the transmission velocity can provide information related to the depth and location of the trachea. Given that sound travels faster through solids than through air, the location of the trachea (an air-filled structure) can be identified. Ultrasonic range finding, or sonar, can also be used to localize the trachea. With this method, an ultrasonic pulse is generated in a known direction. When the ultrasound pulse hits the trachea it will be reflected back to the transmitter as an echo. Using well-known techniques, it is possible to determine the depth of the trachea by measuring the difference in time between the pulse being generated and the echo being received. The speed at which the pulse travels through tissue will depend on the character of the intervening tissue. In some implementations of the present invention, the device is automatically calibrated based on each patient's individual tissue characteristics (e.g., fat content, etc.). In some implementations, a pulse wave is propagated through the neck tissue with a known distance between the emitter and receiver. The distance between the emitter and receiver (if not co-localized), can be determined based on, for example, the known fixed linear distance between the emitter and receiver and the measured curvature of the guidance apparatus, as determined by accelerometer angle data. The velocity of the pulse wave correlates with the character and properties of the intervening tissue. The velocity will be slower in tissue planes that have a higher transmission distance through air (i.e. the trachea). Detection of the trachea using body-penetrating radar operates in an analogous manner.

### User-Interface

In an embodiment, the guidance apparatus of the present invention further comprises one or more LEDs, LCDs 115 or other visual display devices. In such an embodiment, the visual display 115 receives input signals from the microcontroller 105 based on the inputs to the controller 105 from the sensors 110, such that the relative location, depth, orientation, velocity, and/or other parameters of the magnetic intubation stylet 600 can be visually presented to a user. Furthermore, the depth, location, and boundary limits of the trachea and/or other anatomic landmarks can be visually represented through the user-interface. With the teachings described herein, a variety of other visual or auditory signaling methods can be employed, which will be obvious to those skilled in the art. In one embodiment, the apparatus contains an array of LEDs. The brightness and/or color of each LED can indicate the location of the intubation stylet relative to the trachea, such as shown in FIGS. 5A-5D. For example, if the stylet is located lateral to the trachea, the LEDs corresponding to the lateral side of the apparatus can be illuminated, as shown in FIGS. 5A and 5B. As the stylet moves towards the trachea (midline structure), as shown in FIG. 5C, the central LEDs may begin to illuminate while the lateral LEDs get progressively dimmer or change color. The brightness of each LED can also indicate the depth of the stylet with respect to the trachea. For example, as the magnet is positioned deeper to the trachea (more posterior), the LED intensity can decrease. In addition to manipulating the brightness, the color of each LED can also be altered to provide specific feedback to the operator. The LEDs may also flash at a fixed or variable rate to provide additional visual feedback. Any pattern of visual (or auditory) feedback can be employed to facilitate endotracheal intubation, as long as the visual/auditory cues properly represent the relative location, orientation, and trajectory of the intubation stylet with respect to the trachea and can be understood by a user, for example emergency medical personnel.

In some implementations, successful insertion of the magnetic intubation stylet into the trachea is represented by a characteristic light pattern, such as shown in FIG. 5D. When the location of the magnetic intubation stylet is determined to be within the boundaries of the trachea, an indication of successful intubation can be provided to the care provider.

As noted above, in some embodiments, the brightness or color of one or more LEDs can indicate the probability that the stylet has successfully been inserted into the trachea. However, if the stylet is inadvertently placed into the esophagus, the LED(s) may not illuminate, or may be dim, or may change a characteristic color, depending upon the implementation. Any pattern of visual or auditory feedback that provides meaningful information to the care provider can be employed to indicate the probability that the intubating stylet is in the correct location.

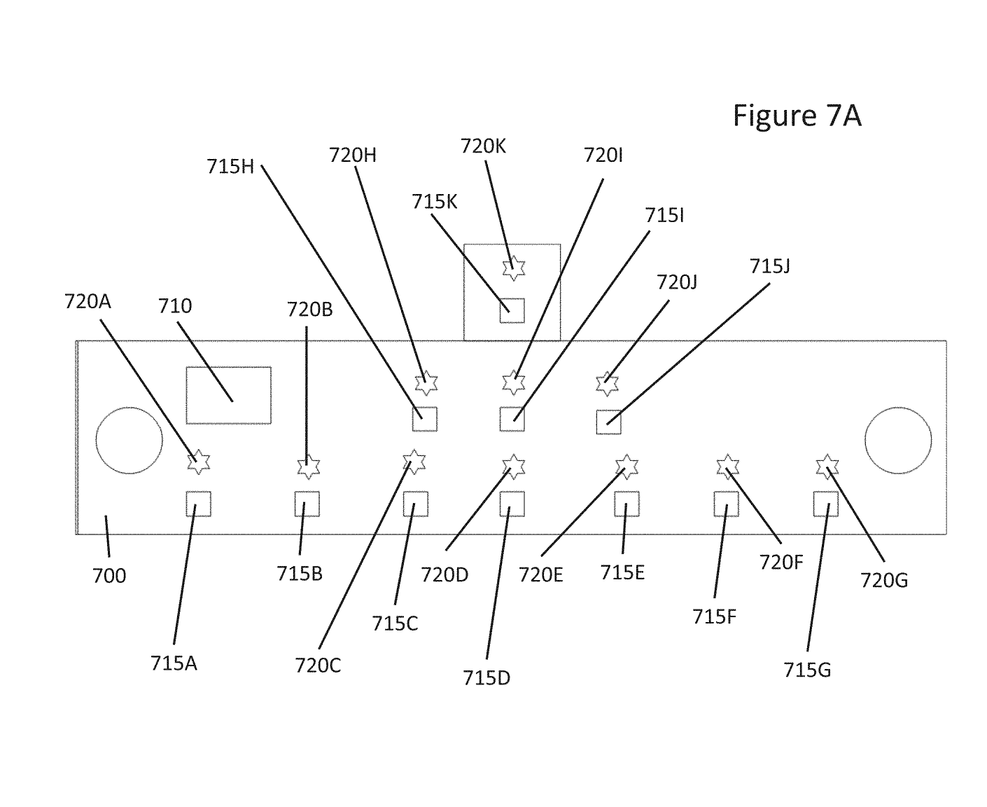

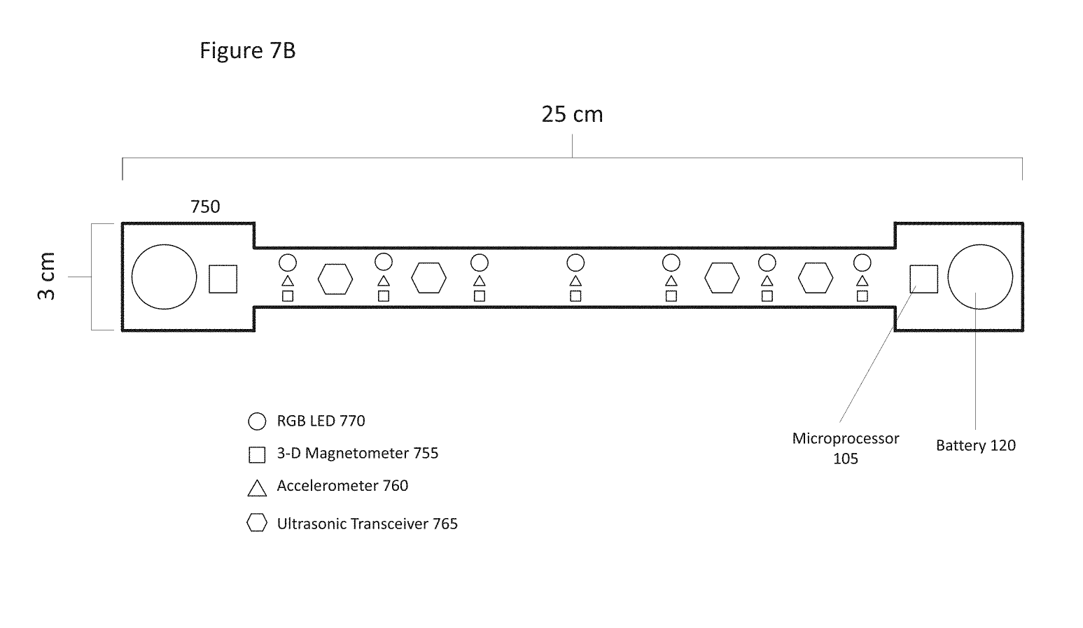

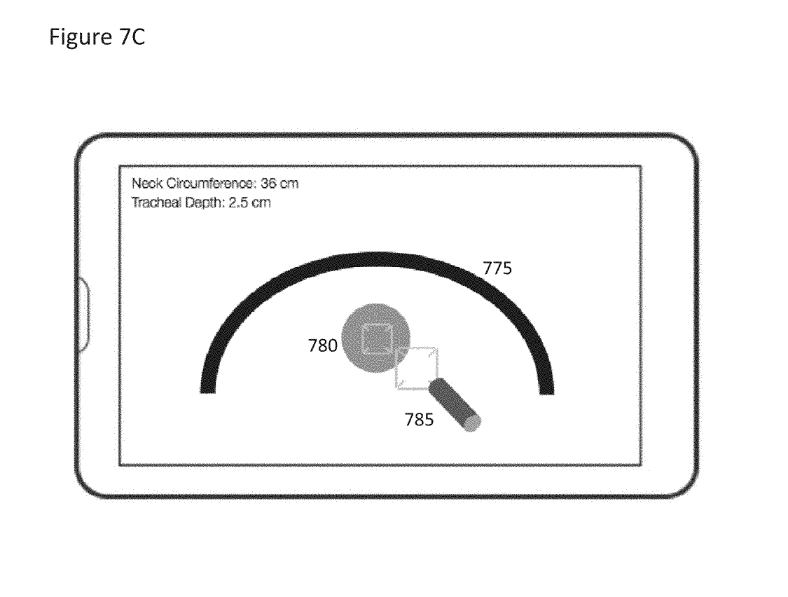

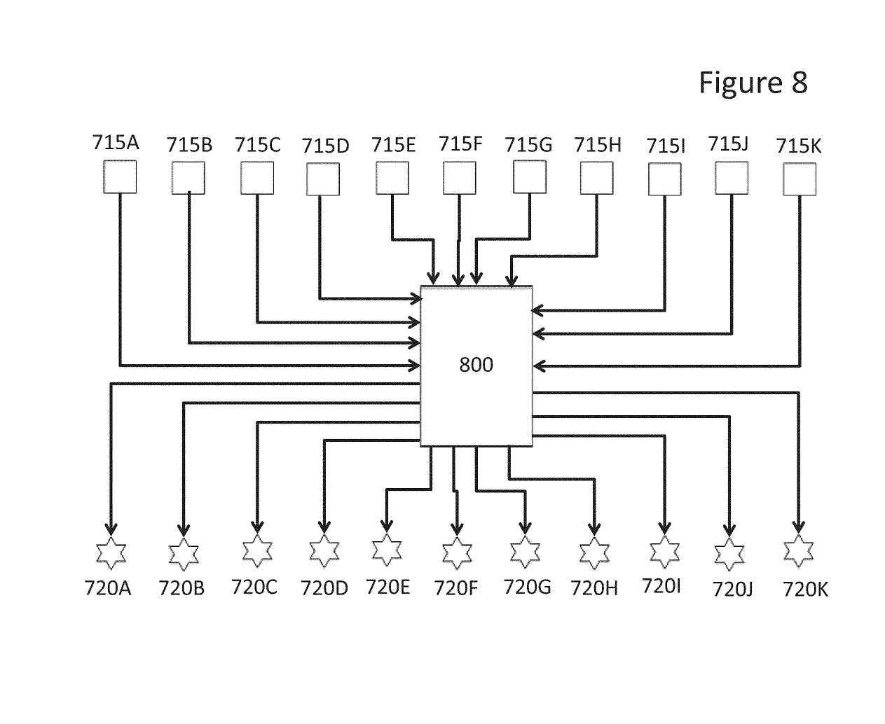

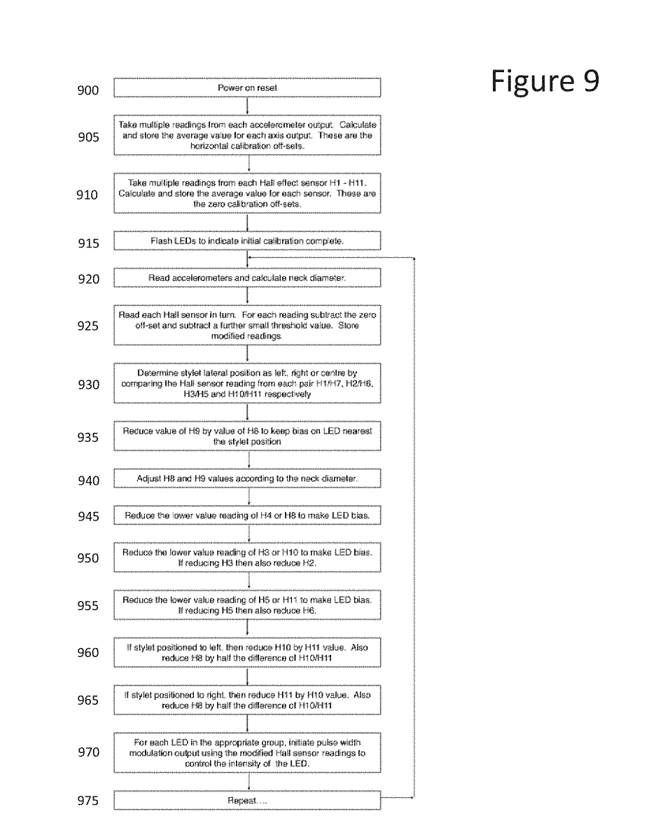

Shown in FIG. 7A, and illustrated in simplified schematic diagram form in FIG. 8, is an alternative embodiment of the guidance device of FIG. 1, in which a more extensive array of Hall sensors and LED's is utilized. In the embodiments of FIGS. 7A and 8, eleven Hall sensors and eleven LED's are arranged in three rows (bottom has seven, middle three, top one), with the Hall sensors providing their inputs to a controller 710 such as an Atmel ATMEGA32U4. The controller 710 calibrates the device as described in FIG. 9, where H1-H11 represent the Hall sensors 715A-715K, respectively, and then monitors the outputs of the Hall sensors to determine the location of the stylet magnet, and, in accordance with the determined position, illuminates the appropriate LEDs 720A-K to guide the user in positioning the stylet into the trachea. As noted previously, the guidance device can also comprise accelerometers, which can, but need not, be implemented in the same chips as the Hall sensors or other magnetometers.

Referring next to FIG. 7B, a still further alternative embodiment of a guidance device in accordance with the present invention can be appreciated. In the embodiment of FIG. 7B, the substrate 750 is flexible and can be comprised of any of the materials described above. Mounted thereon are one or more logic modules 105 comprising controllers or microprocessors and wireless communications logic, as well as one or more batteries 120. The batteries provide power in the normal manner. In addition, mounted on the substrate 750 and in communication with the controller and associated logic substantially as shown in FIG. 8 are a plurality of 3D magnetometers 755, a plurality of accelerometers 760, and a plurality of ultrasonic transceivers 765. The logic 105 processes the data received from the magnetometers, accelerometers and ultrasonic or analogous transceivers at appropriate times and provides an output either to the RGB LEDs 770, or wirelessly to a display such as a smartphone or similar device, or to both. In the embodiment shown in FIG. 7B, the tab shown in FIG. 1 has been removed for improved patient comfort while the device is positioned on the patient's neck. It will be appreciated by those skilled in the art that, if body-penetrating radar is used, such transducers typically replace ultrasound transceivers 765.

The ultrasonic transceivers can be, for example, Steiner & Martins SMATR15F45H5 devices, and can provide additional detail regarding the anatomy of the neck. In general, tissue layers will cast an acoustic shadow on tissue layers located behind them. The degree of shadowing will depend on the characteristics of the tissue layer. The reduction in intensity of the transmitted energy (i.e. shadow effect) can be analyzed and used to define tissue planes. The relative location of specific tissue planes can be determined based on the known relative position/orientation of the ultrasonic transducer and the measured time delay. Increasing the number of ultrasonic transceivers can increase the tissue mapping resolution. In an embodiment of the present invention, the apparatus has one or more ultrasonic transceivers 765 that are located at least partially circumferentially around the patient′ neck. The array of ultrasonic transducers can be used individually, or in combination, to define the cross-sectional anatomy of the patient's neck and determine the location of the trachea. Depending upon the embodiment, the necessary image processing can be performed onboard or the data can be transmitted to an external processor for processing in a manner well-known to those skilled in the art.

In an embodiment of the present invention, the cross-sectional anatomy at specific locations on the neck can be recorded (i.e. via ultrasonic or analogous measurements, bioimpedance measurements, and/or accelerometric measurements). When the user moves the apparatus, such as longitudinally up and down the neck, the cross-sectional anatomy at a variety of locations can be measured and recorded. The exact movement of the apparatus can be determined by the device's accelerometers. In such a fashion, a 3-dimensional tissue map of the neck, particularly related to the airway anatomy, can be obtained. The determined location and trajectory of the trachea can be used to provide an improved airway assessment and can also be used to help facilitate endotracheal intubation when combined with magnetometer data from the magnetic intubation stylet.

In some implementations, data from the guidance apparatus is communicated through a user-interface that displays a visual representation of the magnetic intubation stylet, the anterior neck topography, the trachea, and/or any other anatomic landmarks. In such a fashion, a virtual 2-D or 3-D image of the intubation process can be provided to users, as shown in FIG. 7C. In some implementations, the user-interface is communicated wirelessly to a remote display, such as a computer terminal or mobile device. In other implementations, a wired communication link exists between the guidance apparatus and the user-interface display. In still other implementations, the user-interface is provided locally on the guidance apparatus itself and also communicated via a wired or wireless communication means to a remote display.

In the example of a user-interface as shown on the display of FIG. 7C, the black arc 775 corresponds to the curvature of the guidance apparatus as it rests over the patient's anterior neck. As discussed previously, the curvature of the guidance apparatus can be designed to approximate the curvature of the patient's anterior neck tissue. The gray circle 780 represents the trachea, the depth of which can be determined via accelerometers (neck circumference vs tracheal depth relationship) and/or bio-impedance sensing and/or ultrasonic sensing. In some implementations, the location of the trachea can be represented probabilistically via a heat map, where the color intensity represents the probability that the trachea is in a given location.

As shown in FIG. 7C, a cylinder 785 can represent the position, orientation, and trajectory of the magnetic intubation, as determined by the magnetic sensor array. In order to visually represent the distance between the trachea and intubation stylet, targeting boxes can be provided. For example, as the magnet approaches the trachea, the boxes can get closer together and become the same size and then ultimately be superimposed upon each other as the magnet enters the trachea. Additional ways of visually illustrating the position of the magnetic stylet and the trachea will be obvious to those skilled in the art. In addition to providing visual feedback of the intubation process, in some implementations the UI can display information regarding the patient's anatomy, such as the calculated neck circumference, tracheal depth, or predicted difficulty of intubation (as discussed elsewhere herein).

Once the endotracheal tube has successfully been inserted through the vocal cords and into the trachea, the guidance apparatus can also help provide an indication of the proper depth of the endotracheal tube. It is possible for endotracheal tubes to be placed too deep (such as near the carina or in a bronchus) or too shallow (such as near the vocal cords). The guidance apparatus can help indicate that the endotracheal tube is located in an appropriate position. In some implementations, particularly if the endotracheal tube itself has magnetic properties, the system can ensure that the endotracheal tube is placed at a proper depth within the trachea.

### Magnetic Intubation Stylet

The magnetic intubation stylet, shown in FIG. 6, provides an external magnetic field that can be detected by the guidance apparatus. All or a portion of the stylet may be magnetized. In some implementations, the guidance apparatus is calibrated to work with a magnetic stylet that has a particular size, shape, and magnetic field profile. In some implementations, the stylet has several magnets incorporated that are oriented in different directions. For example, two magnets can be arranged orthogonally to each other, such that their magnetic fields produce a greater sphere of detection. Stated differently, by having magnets oriented in different directions, the distance at which the magnetometers can detect the magnetic field is increased along the sphere of detection (i.e. the radius of detection is increased).

In a preferred embodiment, the magnetic intubation stylet is flexible and conformable, such that it can assume the shape desired by the operator. Some operators may wish to bend the stylet near its distal end and/or proximal end. The stylet should be thin enough such that it can be reversibly inserted inside a standard endotracheal tube. In some implementations, a gum elastic bougie or similar device can be magnetized and detected by the guidance apparatus. In still further implementations, the endotracheal tube itself can be magnetized such that the guidance apparatus can detect it.

In a preferred embodiment, the distal end of the intubation stylet is magnetic. For example, the distal end of the stylet can be composed of a neodymium super strong cylinder magnet that is ⅛″ in diameter and 1″ long. In another embodiment, the entire stylet is magnetic. Those familiar with the art will recognize that there are many possible ways of incorporating a magnet into the intubation stylet, or magnetizing all or a portion of an intubation stylet. As mentioned previously, any device or apparatus that is intended to be inserted into the trachea (such as a bougie, stylet, endotracheal tube, suction catheter, etc.) can be magnetized in a fashion that will enable guidance using the techniques described herein.

### Utilizing Magnetic Force to Facilitate Intubation

In some embodiments, the magnetic aspect of the intubation stylet can move, bend, or rotate with respect to the stylet to which it is attached. For example, a magnet may be attached to the stylet via a hinge mechanism, such that the magnetic aspect can bend independently of the stylet. In some embodiments, the magnetic aspect of the intubation stylet is able to move freely with respect to the stylet. In still other embodiments, the magnetic aspect of the intubation stylet can move in response to an external magnetic field irrespective of the direction or orientation of the intubation stylet to which it is attached. In some implementations, the magnetic aspect of the stylet will always move in concert with the stylet. In such a fashion, in an embodiment of the guidance apparatus which contains an external magnet, and the guidance apparatus is placed over or near the trachea, this external magnet can impose a force on the magnetic intubation stylet to encourage anterior displacement and endotracheal insertion of the stylet. If a magnet were incorporated into the guidance apparatus, the baseline/background magnetic field fingerprint could be subtracted away algorithmically, such that the magnetic field created by the intubation stylet could be detected and guidance provided.

In some implementations, an electromagnet is incorporated into the guidance apparatus, such that the strength of the magnetic field provided by the electromagnet can be modulated. In such a fashion, the magnetic intubation stylet can be physically guided into the trachea using magnetic attraction forces. The guidance apparatus can push or pull the stylet based on the stylet's location relative to the trachea. The strength of magnetic attractive forces applied may vary based on the location of the stylet relative to the trachea.

### Airway Assessment

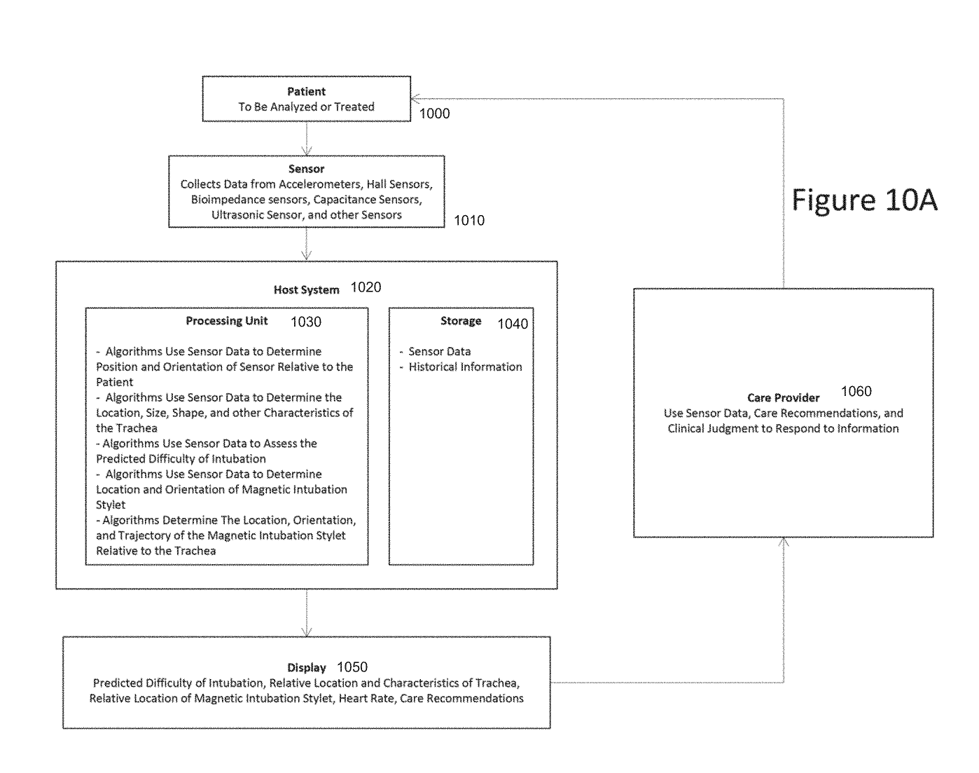

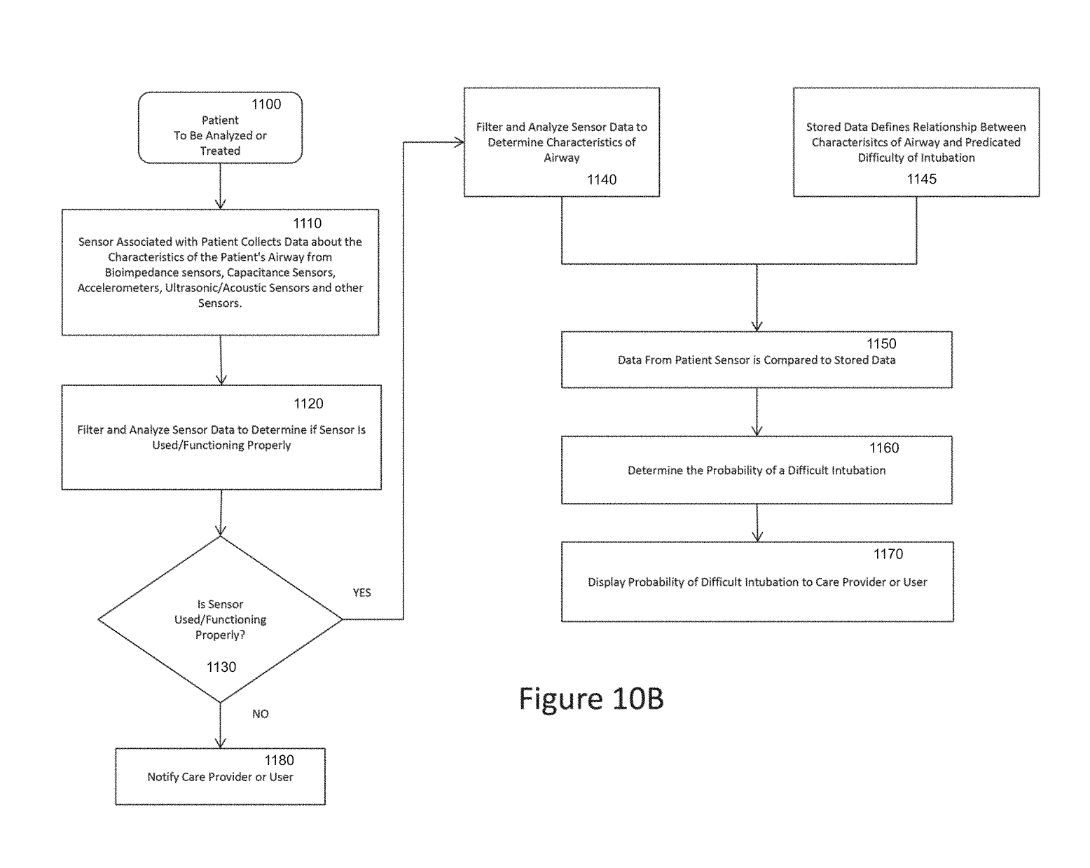

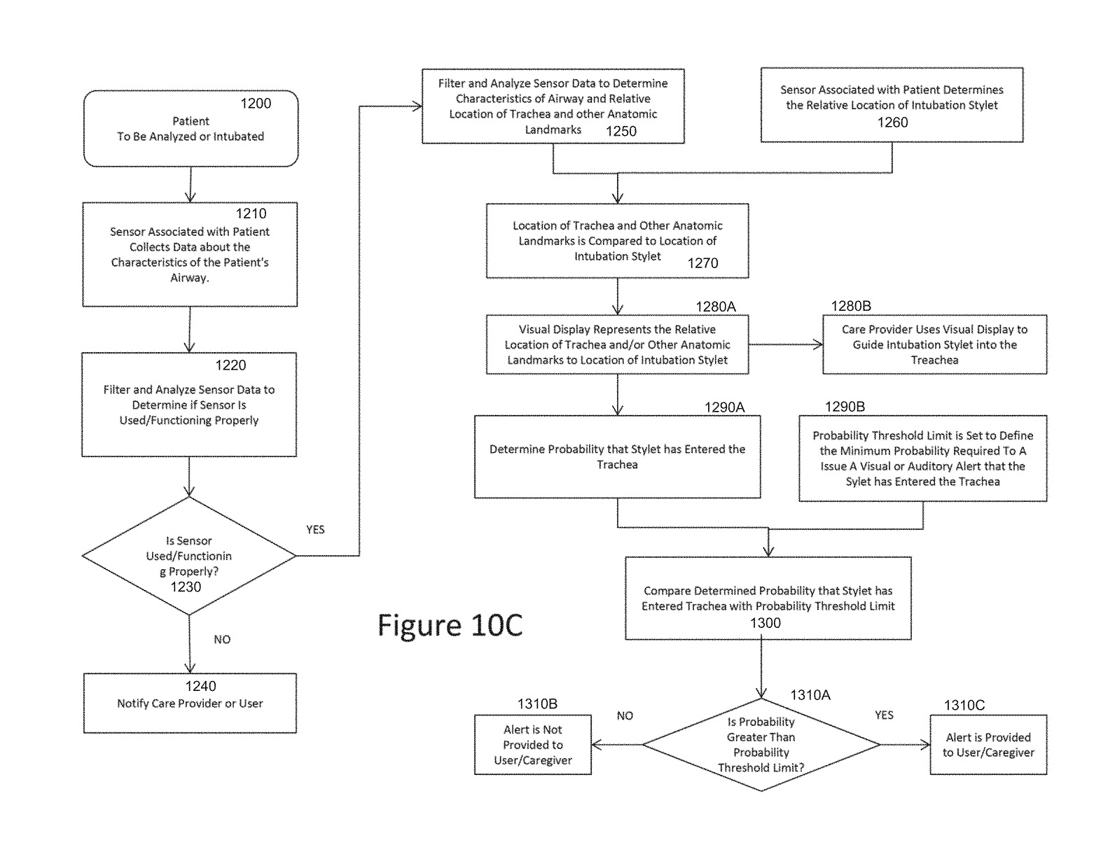

As discussed previously, airway assessment and identifying patient's with a potentially difficult airway is an important component of the physical exam. It is known that there is a correlation between neck circumference and difficulty of intubation. Difficult intubation has also been shown to be more common in obese patients relative to non-obese patients. The relative location of the trachea with respect to the neck (i.e. anterior trachea) may also increase the difficulty of intubation. The system, methods, and devices described herein, and shown in FIGS. 10A-10C, can be used to analyze a patient's airway and provide an indication of the expected difficulty of intubation.

The accelerometers described herein can be used to estimate the circumference of the patient's neck and also estimate tracheal depth. Tissue bioimpedence sensing can also be used to determine the location of the trachea, and also define other anatomic landmarks. Furthermore, bio-impedance sensing can used to define the character, composition, and compliance of the tissues of the neck. Acoustic or ultrasonic sensing can also be used to analyze the neck tissues and determine the relative location of the trachea. These sensing modalities can be used separately or in combination to provide a measure of difficult intubation. In some implementations, the probability of difficult airway is assessed according to accelerometer data, bio-impedance data, and/or ultrasonic [or analogous] data and according to one or more experience-based algorithms. The interaction of these features can be appreciated from FIG. 10A, where the patient to be treated is shown at 1000, and the sensor or sensor array of the present invention is applied to the patient's neck at 1010. At 1020, the host system, which can be a desktop computer, laptop, tablet, smartphone, or similar device, receives the data from the sensor and, using a processor together with one or more of the foregoing sets of algorithms to process the data [shown at 1030], in some cases with stored data and/or historical information as shown at 1040, provides information to a caregiver, such as predicted difficulty of intubation, relative location and characteristics of the trachea, location of the intubation stylet relative to the trachea, and so on. The care provider can then use the displayed information to better treat the patient.

FIG. 10B offers additional refinements, where the patient to be treated is shown at 1100, and the sensor collects data at 1110 as discussed above in connection with FIG. 10A. At 1120, the sensor data is filtered and analyzed either locally or remotely to ensure that the sensor is functioning properly and being used properly. If so, determined at 1130, the sensor data is analyzed as above and, as shown at steps 1140-1170, the caregiver is provided with information to assist in assessing difficulty and performing the intubation.

FIG. 10C further elaborates on the process of FIGS. 10A-10B, with steps 1200-1250 analogous to those in FIG. 10B. In step 1260 the sensor determines the location of the stylet relative to the expected location of the trachea, and a comparison is made at 1270. A visual display is provided to the caregiver at 1280A and used by the caregiver at 1280B. The probability that the stylet has entered the trachea is determined at 1290A, based on a threshold defined at 1290B in reliance on the anatomical characteristics of the patient along with other data from the sensor as discussed above. The probability is then calculated at 1300, and an alert is either sent or not to the caregiver, depending upon the assessed probability, shown at 1310A-C.

### Structure of the Apparatus

In some implementations of the present invention, the apparatus is non-conformable and comes in a variety of pre-set shapes and sizes. The user can select a particular shape/size based on the individual patient's anatomy. In such an implementation, the sensitivity of the magnetic array can be pre-defined based on the shape/size of the apparatus.

If the apparatus is conformable/flexible, the relative orientation of each magnetometer may change. It may be desirable to know the orientation of each magnetometer with respect to each other or to the environment. There are a variety of ways of providing data regarding the orientation of each magnetometer. In a preferred embodiment of the present invention, the apparatus may contain one or more accelerometers, such that the arc or curvature of the apparatus (and thus the associated magnetometers) can be determined. Alternatively, each magnetometer can be individually associated with an accelerometer. In such a fashion, the relative orientation of the magnetometers with respect to the environment can be determined. Based on the relative orientation of each Hall sensor, the device can be automatically calibrated and thus customized based on the individual patient's anatomy.

In some implementations, the apparatus may have a removable component that is designed to contact the patient and then be disposed of following use of the apparatus. In such a fashion, the sterility of the device can be maintained between patients by exchanging the removable patient-contacting component. In some implementations, the removable component may have an adhesive quality to improve skin contact. The removable component may also have conductive properties, which can in some implementations improve bio-impedance sensing.

A variety of other sensing modalities can be incorporated into the apparatus to provide an improved automatic calibration means. For example, capacitance or thermal sensors can be used to determine the degree of continuity/contact between the apparatus and the patient's skin. In some implementations, feedback can be provided to the operator if the apparatus is not adequately in contact with the patient's skin. Stretch and/or strain receptors can also be incorporated into the apparatus to provide information regarding the shape of the flexible apparatus in real-time.

Given that the guidance apparatus is designed to detect the location of external magnetic fields, in some implementation it may be necessary to zero out geomagnetic fields or other extraneous magnetic fields that may be present in the environment. In some implementations, when the guidance apparatus is placed on the patient, all magnetic fields are algorithmically zeroed out. A visual or auditory indication can be provided to indicate when the guidance apparatus has been magnetically zeroed within the environment. Any subsequent magnetic fields detected can be assumed to be originating from the magnetic guidance apparatus.

The guidance apparatus may communicate to an external host via wireless or wired means. From the host system, data obtained from the guidance apparatus can be displayed on external monitors, cell phone, tablet, or other means. In addition, data obtained from the guidance apparatus can be overlayed or used in conjunction with video laryngoscopy imaging.

Having fully described a preferred embodiment of the present invention and various alternatives thereof, those skilled in the art will recognize that variations exist that do not depart from the teachings described herein and are intended to be encompassed by the appended claims.

I claim:

1. A sensor array for estimating the location of a patient's trachea comprising a plurality of sensors arranged substantially along the length of a substrate, the substrate being sufficiently flexible to substantially conform to the front portion of the neck of a patient, the sensors comprising at least one of a group comprising ultrasound sensors and Hall sensors, and
a processor for receiving data from at least some of the sensors for assessing the probable location of a patient's trachea and for providing an output suitable for display to a caregiver.

2. A sensor array for detecting the presence of a magnetic stylet for intubation of a patient comprising
a substrate having arranged thereon a plurality of sensors for detecting the proximity of a magnetic stylet movably positioned within the throat of a patient to be intubated, the substrate being sufficiently flexible to substantially conform to a front portion of the neck of the patient,
a processor for receiving signals or data from the plurality of sensors and determining the approximate location of the stylet within the throat of the patient a plurality of display devices arranged on the substrate and responsive to inputs from the processor to indicate to a caregiver the approximate location of the stylet relative to the trachea of the patient.
